(19) United States
(12) Patent Application Publication
(10) Pub. No.: US 2025/0366726 A1
(43) Pub. Date: Dec. 4, 2025

(54) WRIST BLOOD PRESSURE MEASURING DEVICE

(71) Applicant: K-JUMP HEALTH CO., LTD., New Taipei City (TW)

(72) Inventor: Chao-Man TSENG, New Taipei City (TW)

(21) Appl. No.: 18/731,770

(22) Filed: Jun. 3, 2024

(51) Int. Cl. A61B 5/0225; A61B 5/00; A61B 5/022; A61B 5/024

(52) U.S. Cl. CPC A61B 5/0225; A61B 5/02233; A61B 5/024; A61B 5/742; A61B 2562/0204; A61B 2562/0219

(57) ABSTRACT

A wrist blood pressure measuring device wore on a wrist of a user comprises a wearable pressure component, a posture sensing unit, a sound sensing unit, a processing unit, an inflation and deflation unit, a pressure sensing unit and an output unit. The posture sensing unit is configured to detect whether the inclination angle of the wrist of the user is between 24° and 26°, the sound sensing unit is configured to detect a heart sound of the user, the processing unit is coupled to the posture sensing unit and the sound sensing unit and outputs a signal associated with the inclination angle and the heart sound, the inflation and deflation unit is configured to inflate a filling gas to an airbag unit based on the signal so that the wearable pressure component exerting a pressure on the wrist.

### FIELD OF THE INVENTION

The invention relates to a blood pressure measuring device, and in particular to a wrist blood pressure measuring blood device for measuring blood pressure in a correct position.

### BACKGROUND OF THE INVENTION

With the rise of health awareness, people have gradually become accustomed to monitoring their own health-related values in their daily lives, as well as detecting potential health problems early, and allowing the doctor to refer to their usual body data during medical appointments to facilitate the doctor's diagnosis.

Sphygmomanometer is one of the common household medical equipment in people's home life. The traditional arm-type sphygmomanometer is relatively bulky. Although the measurement value is accurate, it is not easy to carry and install in various homes. In order to facilitate users to regularly monitor their own blood pressure, a wrist-type sphygmomanometer was developed, which can be worn directly on the user's wrist for measurement. It has the advantages of small size, easy storage and portability.

Generally speaking, the cuff of a conventional arm-type sphygmomanometer is worn on the user's arm, and the user takes measurements in a sitting position with the user's elbow leaning on the table, which ensures the height, angle and distance of the arm in relation to the heart are measured. However, the wrist-type sphygmomanometer is worn directly on the user's wrist, and the posture is not as stable as that of the arm-type sphygmomanometer, which is prone to the problem of inaccurate blood pressure measurement results due to incorrect posture of the measurement. Therefore, it is known that U.S. Pat. No. 9,895,084B2 discloses a wrist-type sphygmomanometer, which can measure blood pressure after confirming the inclination angle and height of the user's wrist. However, the aforementioned device is unable to determine the distance between the measurement position and the heart, and there is still a problem that the measurement data is not accurate enough.

### BRIEF DESCRIPTION OF THE DRAWINGS

FIG. 1 is a schematic diagram of an embodiment of the present invention.

FIG. 2 is a schematic diagram of a method of an embodiment of the present invention.

FIG. 3A is a schematic diagram of use an embodiment of the present invention.

FIG. 3B is a schematic diagram of use an embodiment of the present invention.

FIG. 3C is a schematic diagram of use an embodiment of the present invention.

### SUMMARY OF THE INVENTION

A main object of the invention is to solve the problem that conventional wrist blood pressure measuring device cannot ensure that the user's wrist is in the correct position to measure blood pressure, resulting in inaccurate measurement data.

In order to achieve the above object, the invention provides a wrist blood pressure measuring device worn on a wrist of a user. The wrist blood pressure measuring device comprises a wearable pressure component, a posture sensing unit, a sound sensing unit, a processing unit, an inflation and deflation unit, a pressure sensing unit and an output unit. The wearable pressure component is worn on the wrist and comprises an airbag unit and a wearing unit for fixing the airbag unit on the wrist of the user. The posture sensing unit is configured to detect whether an inclination angle of the wrist of the user is between 24° and 26°. The sound sensing unit is configured to detect a heart sound of the user. The processing unit is coupled to the posture sensing unit and the sound sensing unit and outputs a signal associated with the inclination angle and the heart sound. The inflation and deflation unit is configured to inflate a filling gas to an airbag unit based on the signal so that the wearable pressure component exerting a pressure on the wrist.

### DETAILED DESCRIPTION OF THE PREFERRED EMBODIMENTS

The terminology used herein is only for a purpose of describing specific embodiments and does not intend to limit the invention. As used herein, the singular forms “a”, “an” and “the” may include the plural forms as well, unless the context dictates otherwise.

Directional terms used herein, such as up, down, left, right, front, back and their derivatives or synonyms, refer to the orientation of elements in the drawings and do not limit the invention unless the context clearly indicates otherwise.

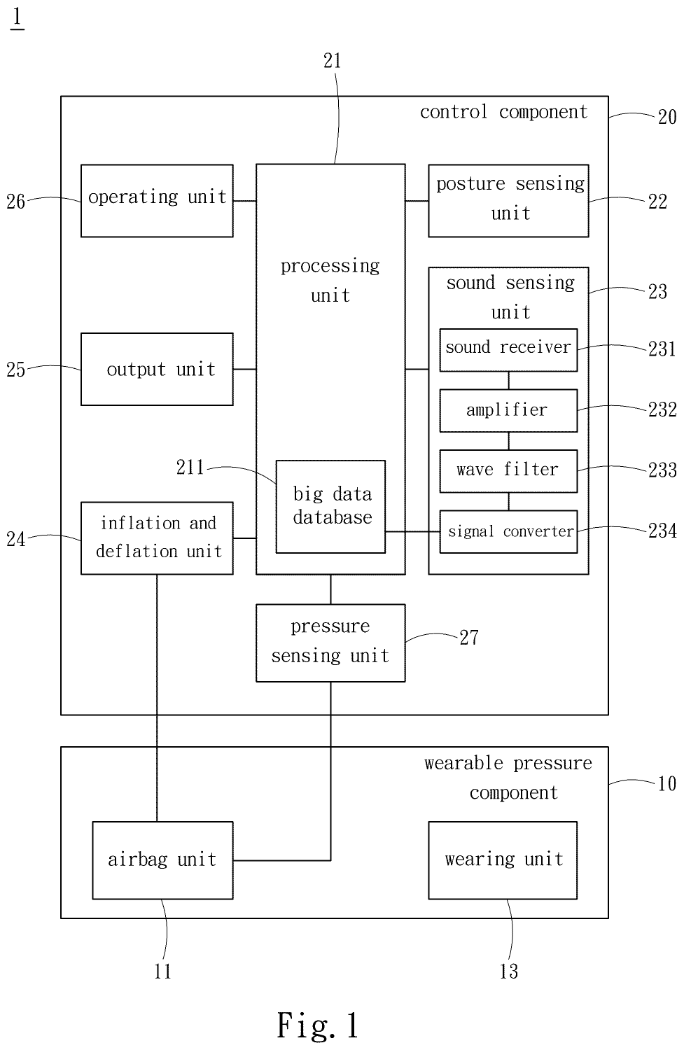

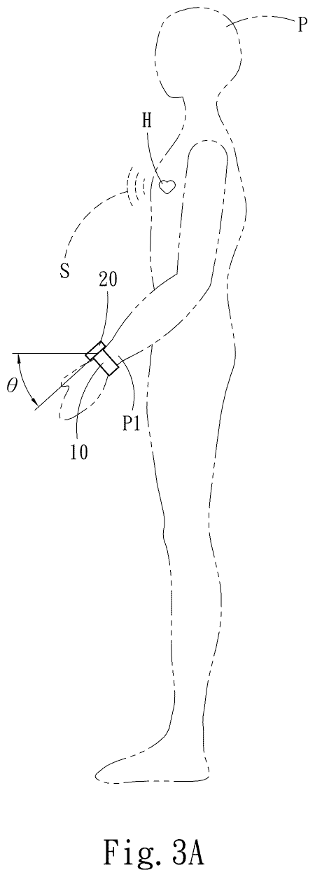

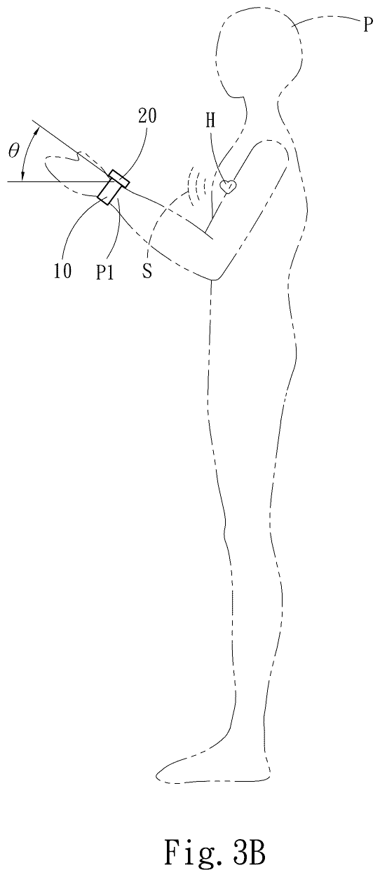

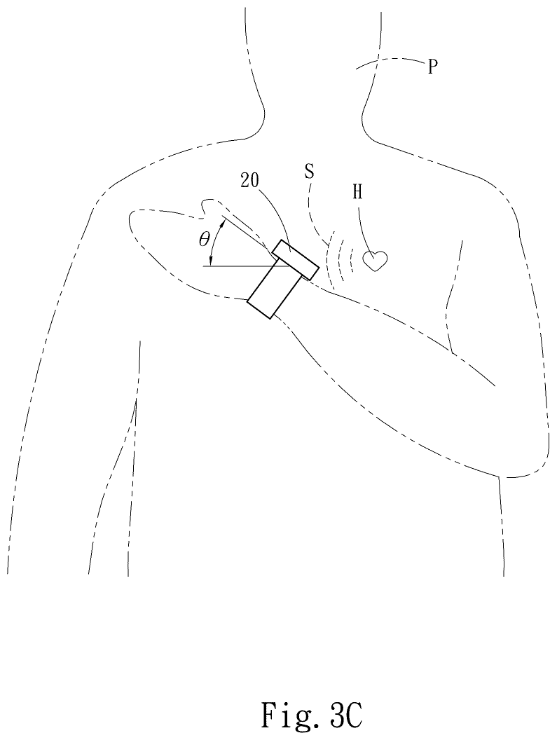

Please refer to FIG. 1, FIG. 3A, FIG. 3B and FIG. 3C. The invention provides a wrist blood pressure measuring device 1 worn on a wrist P1 of a user P. The wrist blood pressure measuring device 1 includes a wearable pressure component 10 and a control component 20. The wearable pressure component 10 is coupled to the control component 20 so that the control component 20 is fixed to the wearable pressure component 10, which can be easily carried as a whole. In this embodiment, the wearable pressure component 10 is an inflatable wristband, and the control component 20 is a device host used to control inflation and deflation of the wearable pressure component 10 and to display physiological values, such as systolic pressure, diastolic pressure, pulse, and heart rate, etc., measured from the user P.

The wearable pressure component 10 includes an airbag unit 11 and a wearing unit 13. The wearing unit 13 is disposed on the airbag unit 11 and is worn on the wrist P1 of the user P to fix the wearable pressure component 10. In one embodiment, the wearing unit 13, such as Velcro, buckles, etc., can be used to remove and wear the wearable pressure component 10 repeatedly.

The control component 20 includes a processing unit 21, a posture sensing unit 22, a sound sensing unit 23, an inflation and deflation unit 24, an output unit 25, an operating unit 26 and a pressure sensing unit 27. The processing unit 21 is coupled to the posture sensing unit 22, the sound sensing unit 23, the inflation and deflation unit 24, the output unit 25, the operating unit 26 and the pressure sensing unit 27. The processing unit 21 is used to receive, send signals or instructions. The pressure sensing unit 27 is coupled to the airbag unit 11 and used to detect a pressure in the airbag unit 11. When the airbag unit 11 is in an inflated state to press blood vessels in the wrist P1 of the user P, the pressure sensing unit 27 senses a pressure according to a pulse in the blood vessels in the wrist P1.

The posture sensing unit 22 is provided for detecting a wrist posture of the user P. Specifically, the posture sensing unit 22 is provided for detecting an inclination angle θ of the wrist P1 of the user P. Taking the wrist blood pressure measuring device 1 worn on the wrist P1 of a left hand of the user P as an example, FIG. 3A and FIG. 3B are viewed from a left side of the user P, FIG. 3C is viewed from a front side of the user P, a horizontal plane is defined as a reference in FIG. 3A, FIG. 3B and FIG. 3C, and the inclination angle θ is positive when the inclination angle θ is a clockwise angle, and the inclination angle θ is negative when the inclination angle θ is a counterclockwise angle. In one embodiment, the posture sensing unit 22 is an acceleration sensor and is configured to detect whether the inclination angle θ of the wrist P1 of the user P is between 24° and 26°.

The sound sensing unit 23 is provided for detecting a heart sound S of the user P. The processing unit 21 outputs a signal associated with the inclination angle θ and the heart sound S to the inflation and deflation unit 24. The inflation and deflation unit 24 is connected to the airbag unit 11 and provides a filling gas to the airbag unit 11 based on the signal, and the filling gas is air. The inflation and deflation unit 24 also can discharges the filling gas in the airbag unit 11. The output unit 25 is provided for displaying a measurement result and/or making a sound. The operating unit 26 is provided for controlling the processing unit 21. In one embodiment, the sound sensing unit 23 is a microphone, the inflation and deflation unit 24 is a pump, the output unit 25 is a display screen, a speaker, or a combination of the above, and the operating unit 26 is an operation button and a power switch. In other embodiments, the output unit 25 and the operating unit 26 are integrated into a touch screen for operation and displaying the measurement result.

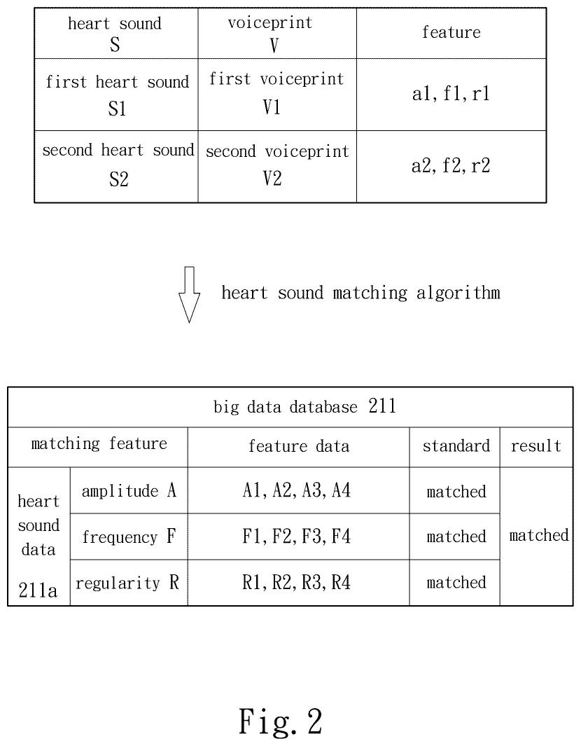

Please refer to FIG. 1 and FIG. 2, the processing unit 21 includes a big data database 211 coupled to the sound sensing unit. The big data database 211 is associated with a plurality of heart sound data 211a. The plurality of heart sound data 211a include an amplitude A, a frequency F, a regularity R, or a combination of the above.

The sound sensing unit 23 includes a sound receiver 231, an amplifier 232, a wave filter 233, and a signal converter 234. The amplifier 232 is coupled to the sound receiver 231, the wave filter 233 is coupled to the amplifier 232, the signal converter 234 is coupled to the wave filter 233, and the signal converter 234 is connected to the big data database 211. The heart sound S of the user P is detected by the sound receiver 231 and passes through the amplifier 232, the wave filter 233, and the signal converter 234 in sequence, and is compared by the big data database 211.

A volume of the heart sound S is increased in the amplifier 232, and specific frequency of sounds is removed by the wave filter 233, and the signal converter 234 converts the heart sound S from analog to digital so that a voiceprint V of the heart sound S of the user P is obtained by converting a continuous signal in analog form to a discrete signal in digital form. A heart sound matching algorithm determines whether the heart sound S meets a standard by matching the voiceprint V and the plurality of heart sound data 211a in the big data database 211. The standard is set to determine the similarity or consistency of the voiceprint V of the heart sound S of the user P based on the amplitude A, the frequency F, the regularity R of the plurality of heart sound data 211a. In one embodiment, the frequency F of the plurality of heart sound data 211a is between 20 Hz and 200 Hz.

Further, the heart sound S of the user P includes a first heart sound S1 and a second heart sound S2, and the first heart sound S1 and the second heart sound S2 respectively include a first voiceprint V1 and a second voiceprint V2. In addition, the first voiceprint V1 includes an amplitude feature a1, a frequency feature f1 and a regularity feature r1, and the second voiceprint V2 includes an amplitude feature a2, a frequency feature f2 and a regularity feature r2.

The amplitude feature a1, the frequency feature f1, and the regularity feature r1 of the first voiceprint V1, and the amplitude feature a2, the frequency feature f2, and the regularity feature r2 of the second are respectively compared with multiple feature data A1, A2, A3, A4, F1, F2, F3, F4, R1, R2, R3, R4 of the big data database 211 by the heart sound matching algorithm, so as to determine whether the heart sound S meets the standard and obtains a result. If the standard is met, the result is “matched”, which means that the heart sound S is detected; if the standard is not met, the result is “not matched”, which means that the heart sound is not detected. In one embodiment, the frequency feature f1 is between 40 Hz and 60 Hz, and the frequency feature f2 is between 40 Hz and 100 Hz, such that the standard of the feature data F1, F2, F3, F4 are met.

Please refer to FIG. 3A, FIG. 3B, and FIG. 3C, when the user P wants to measure blood pressure, the wearing unit 13 of the wearable pressure component 10 is worn on the wrist P1 of the user P. However, if a position of the wrist P1 is changed by following a movement of the user P, and the wrist blood pressure measuring device 1 cannot accurately measure blood pressure. Specifically, if the inclination angle θ of the posture sensing unit 22 does not meet an angle threshold set by the processing unit 21, or the heart sound S detected by the sound sensing unit 23 does not meet the feature data A1, A2, A3, A4, F1, F2, F3, F4, R1, R2, R3, R4 set by the processing unit 21 (i.e., any one of the inclination angle θ and the heart sound S does not meet preset conditions), the processing unit 21 controls the inflation and deflation unit 24 not inflate the airbag unit 11. In one embodiment, the angle threshold is between 24° and 26°.

For example, when the wrist P1 of the user P is in a position such as shown in FIG. 3A, the inclination angle θ does not meet the angle threshold set by the processing unit 21, and the heart sound S is not detected by the sound sensing unit 23. Under this situation, the inflation and deflation unit 24 does not inflate the airbag unit 11. In another example, when the wrist P1 of the user P is in a position such as shown in FIG. 3B, even if the inclination angle θ of the wrist P1 meets the angle threshold (the inclination angle θ is between 24° and) 26° set by the process unit 21, however, the wrist P1 is far from a heart H of the user P so that the sound sensing unit 23 cannot detect the heart sound S, and the inflation and deflation unit 24 does not inflate the airbag unit 11 at the time.

When the wrist P1 of the user P is raised to a position parallel to the heart H and close to the heart H (as shown in FIG. 3C), the inclination angle θ is within a range of the angle threshold, and the heart sound S meets the feature data A1, A2, A3, A4, F1, F2, F3, F4, R1, R2, R3, R4 (i.e., the inclination angle θ and the heart sound S meet the preset conditions), the processing unit 21 controls the inflation and deflation unit 24 inflate the airbag unit 11 to measure blood pressure.

Further, the inclination angle θ detected by the posture sensing unit 22 is in a range of 24° to 26°, and the sound sensing unit 23 detects the heart sound S, the wrist blood pressure measuring device 1 performs the following steps:
S1: inflating the filling gas to the airbag unit 11 of the wearable pressure component 10 by the inflation and deflation unit 24 so that the wearable pressure component 10 worn on the wrist P1 exerts the pressure on the wrist P1.
S2: transmitting the pressure detected by the pressure sensing unit 27 to the processing unit 21, and the processing unit 21 generates a blood pressure data based on the pressure.
S3: detecting the inclination angle θ is not in the range of 24° to 26° by the posture sensing unit 22, or the heart sound S is not detected by the sound sensing unit 23 (either condition is met), the wrist blood pressure measuring device 1 is in a non-activated state, that is, the processing unit controls the inflation and deflation unit 24 not to inflate the airbag unit 11, and the output unit 25 does not display the measurement result.

According to the above disclosure, the wrist blood pressure measuring device of the invention includes the posture sensing unit and the sound sensing unit at the same time. When the inclination angle detected by the posture sensing unit and the heart sound detected by the sound sensing unit meet the preset conditions, the processing unit controls the inflation and deflation unit to inflate the filling gas to the airbag to perform blood pressure measurement steps, which can avoid blood pressure measurement in wrong postures, causing inaccurate blood pressure measurement result.

What is claimed is:

1. A wrist blood pressure measuring device, worn on a wrist of a user, the wrist blood pressure measuring device comprising:
a wearable pressure component, comprising an airbag unit and a wearing unit for fixing the airbag unit to the wrist of the user;
a posture sensing unit, configured to detect whether an inclination angle of the wrist of the user being between 24° and 26°;
a sound sensing unit, configured to detect a heart sound of the user;
a processing unit, coupled to the posture sensing unit and sound sensing unit, the processing unit outputting a signal associated with the inclination angle and the heart sound;
an inflation and deflation unit, connected to the airbag unit, and configured to inflate a filling gas to the airbag unit based on the signal so that the wearable pressure component exerting a pressure on the wrist;
a pressure sensing unit, coupled to the airbag unit, and configured to transmit the pressure detected to the processing unit, and the processing unit generating a blood pressure data; and
an output unit, coupled to the processing unit, and configured to output a measurement result associated to the blood pressure data.

2. The wrist blood pressure measuring device as claimed in claim 1, wherein the posture sensing unit is an acceleration sensor.

3. The wrist blood pressure measuring device as claimed in claim 1, wherein the sound sensing unit is a microphone.

4. The wrist blood pressure measuring device as claimed in claim 1, wherein the inflation and deflation unit is a pump.

5. The wrist blood pressure measuring device as claimed in claim 1, wherein the output unit is a display screen, a speaker, or a combination of the above.

6. The wrist blood pressure measuring device as claimed in claim 1, wherein the blood pressure data comprises systolic pressure, diastolic pressure, pulse, and heart rate.

7. The wrist blood pressure measuring device as claimed in claim 1, wherein the posture sensing unit detects the inclination angle not in a range of 24° to 26°, the wrist blood pressure measuring device is in a non-activated state.

8. The wrist blood pressure measuring device as claimed in claim 1, wherein the sound sensing unit performs a heart sound matching algorithm to determine whether the heart sound is detected based on a big data database associated with a plurality of heart sound data.

9. The wrist blood pressure measuring device as claimed in claim 1, wherein the heart sound is not detected by the sound sensing unit, the wrist blood pressure measuring device is in a non-activated state.
